(19) United States
(12) Patent Application Publication
(10) Pub. No.: US 2011/0288436 A1
(43) Pub. Date: Nov. 24, 2011

(54) Materials and methods for insulating electronic components and services

(75) Inventor: Robert T. Stone, Sunnyvale, CA (US)

(73) Assignee: Tronics MedTech

(21) Appl. No.: 13/067,000

(22) Filed: Apr. 28, 2011

Related U.S. Application Data
(60) Provisional application No. 61/396,097, filed on May 20, 2010.

(51) Int. Cl. A61B 5/00; B05D 5/12

(52) U.S. Cl. 600/561; 427/58

(57) ABSTRACT

A method for insulating an electronic component, comprising encapsulating the electronic component with an electrically insulating pouch having a first protective layer and a second electrically insulating layer, the first protective layer comprising a flexible polymeric material, the second electrically insulating layer comprising the parylene layer.

### CROSS-REFERENCE TO RELATED APPLICATIONS

This application claims the benefit of U.S. Provisional Application No. 61/396,097, filed May 20, 2010.

### FIELD OF THE PRESENT INVENTION

The present invention relates generally to methods for insulating electronic components and devices. More particularly, but not by way of limitation, the invention relates to materials and methods for insulating active and passive electronic components and devices, whereby the electronic components and devices are suitable for prolonged direct body fluid exposure.

### BACKGROUND OF THE INVENTION

In medical diagnosis and treatment of a living body, it is often necessary to measure and/or monitor one or more physiological characteristics associated with the body. As is well known in the art, several significant physiological characteristics are assessed via one or more pressures within the body.

Various electronic systems and devices have thus been developed to measure and/or monitor pressures within a body. The noted systems and devices include various external and internal (i.e. implantable) pressure sensor systems and devices. Examples of pressure sensor applications include, without limitation, cerebro-spinal fluid pressure, intracranial pressure, intraocular pressure, intratracheal or respiratory pressure, arterial pressure, and urinary bladder pressure.

As is well known in the art, MEMS pressure sensors are particularly usefully for measuring and/or monitoring pressures within a body; particularly, within organs or systems in the body. Illustrative are the MEMS pressure sensor systems and devices disclosed in Co-Pending application Ser. Nos. 12/655,405 and 12/799,752; which are incorporated by reference herein in their entirety.

As is also well known in the art, most implantable pressure sensor systems and devices; particularly, MEMS systems and devices, require absolute hermeticity to provide reliable and consistent functionality during prolonged pressure measurement and/or monitoring. Various systems and methods have thus been developed to hermetically seal pressure sensor systems and devices and, hence, electronic components contained therein.

In the MEMS pressure sensor systems and devices disclosed in Co-Pending application Ser. Nos. 12/655,405 and 12/799,752, a totally hermetic seal (i.e. titanium metal and ceramic feed-through) is provided, which prevents moisture (even in a monomolecular state) from diffusing through the sensor membrane or housing and ultimately degrading the performance of the MEMS pressure sensor.

Various additional methods have also been employed to hermetically seal electronic components that are employed to construct implantable systems and devices. Such methods include coating selective electronic components with an adjunct sealant, such as a polymeric material. Exemplar polymeric materials include polyethylene, polyvinylchloride, polyimide, epoxy and polyurethane. Illustrative are the methods disclosed in U.S. Pat. Pub. Nos. 2008/0242768 and 2009/0305062.

There are, however, several drawbacks and disadvantages associated with coating electronic components of implantable systems and devices with conventional adjunct sealants, such as a polymeric material. Among the drawbacks and disadvantages are poor and/or degradation of adhesion and permeability of some adjunct sealants. Thus, over extended periods of time, moisture can and, in many instances, will still penetrate through most, if not all, conventional adjunct sealants and eventually degrade the performance and/or functionality of any electronic component coated therewith.

Further, adjunct sealants and coatings typically have a different thermal coefficient of expansion as the coated electronic component. Thus, after exposure to multiple temperature excursions or simply after an extended period of time, the adhesion of an adjunct sealant to an electronic component starts to break down.

Additional drawbacks associated with coating electronic components with conventional adjunct sealants stem from the notion that adding an adjunct sealant on an electronic component will allow the component to maintain a high level of functionality in the presence of body fluids.

As is well known in the art, body fluid is an extremely corrosive and conductive medium. There are many dissolved minerals in body fluid, including salt and potassium, which readily conduct electricity in their ionic state.

One of the most common and severe failure of electronic components exposed to bodily fluids thus results from a process known as metal migration, whisker formation or dendritic growth. In the presence of moisture; particularly, a body fluid, metal migration or dendritic growth often occurs; particularly, in areas of opposite polarity on an electronic component, e.g., a capacitor. Such metal migration or dendritic growth can, and in many instances will, lead to either immediate or latent catastrophic failure of the electronic component and device associated therewith.

It would thus be a significant advancement in the art to provide implantable passive and active electronic components and devices that are suitable for prolonged direct body fluid exposure.

It would also be desirable to provide implantable pressure sensors, and associated systems and methods, which provide accurate and stable sensor output over extended periods of time.

It is therefore an object of the present invention to provide materials and methods for insulating implantable passive and active electronic components and devices, whereby the electronic components and devices are suitable for prolonged direct body fluid exposure.

It is another object of the present invention to provide implantable medical systems and devices that are suitable for prolonged direct body fluid exposure.

### SUMMARY OF THE INVENTION

In accordance with the above objects and those that will be mentioned and will become apparent below, the method for insulating an electronic component generally comprises substantially fully encapsulating the electronic component with an electrically insulating pouch, the electrically insulating pouch comprising a first protective layer and a second electrically insulating layer, the first protective layer comprising the flexible polymeric material, the second electrically insulating layer comprising the parylene layer.

In certain embodiments, the electrically insulating pouch includes encasement means.

In certain embodiments, the flexible polymeric material also comprises an electrically insulating layer.

In certain embodiments, the flexible polymeric material comprises a flexible polymeric gel.

In certain embodiments, the flexible polymeric gel comprises silicone.

In certain embodiments, the minimum thickness of the flexible polymeric material is in the range of approximately 10-500 microns.

In certain embodiments, the maximum thickness of the flexible polymeric material is in the range of approximately 50-1000 microns.

In certain embodiments, the thickness of the electrically insulating layer is in the range of approximately 1-10 microns.

In accordance with another embodiment of the invention, there is provided an implantable electronic component that is suitable for prolonged direct body fluid contact, the electronic component including an electrically insulating pouch, the insulating pouch substantially fully encapsulating the electronic component, the insulating pouch comprising a first protective layer and a second electrically insulating layer, the first protective layer comprising the flexible polymeric material, the second electrically insulating layer comprising the parylene layer.

In accordance with another embodiment of the invention, there is provided a pressure sensor system comprising a sensor assembly having (i) at least one MEMS pressure sensor having a pressure sensing element that is adapted to generate a capacitance variation signal in response to exposed pressure, (ii) an application-specific integrated circuit (ASIC) that is adapted to generate a pressure signal with the capacitance variation signal, the pressure signal being representative of the exposed pressure, and (iii) network circuitry in communication with the MEMS sensor and the ASIC, the MEMS sensor, ASIC and at least a portion of said circuitry being substantially fully encapsulated with an electrically insulating pouch, the insulating pouch comprising a first protective layer and a second electrically insulating layer, the first protective layer comprising the flexible polymeric material, the second electrically insulating layer comprising the parylene layer.

In certain embodiments of the invention, the pressure sensor system includes (i) a temperature compensation system that is adapted to correct for temperature induced variations in the pressure sensor signal, (ii) a drift compensation system that is adapted to correct for pressure and temperature induced drift of the pressure sensor signal, and (iii) a pressure compensation system that is adapted to correct for variations in measured pressures of the MEMS pressure sensor and atmospheric pressure.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic, cross-sectional view of a MEMS pressure sensor module, an ASIC Module and electronic circuitry therebetween, the MEMS pressure sensor module, ASIC Module and electronic circuitry being substantially fully encapsulated with one embodiment of an electrically insulating pouch, in accordance with the invention;

FIG. 2 is another schematic, cross-sectional view of the MEMS pressure sensor module, ASIC Module and electronic circuitry, wherein the MEMS pressure sensor module, ASIC Module and electronic circuitry are substantially fully encapsulated with another embodiment of an electrically insulating pouch, in accordance with the invention;

FIG. 3 is a partial cross-sectional, front plane view of one embodiment of an improved pressure sensor, in accordance with one embodiment of the invention; and

FIG. 4 is a cross-sectional, front plane view of one embodiment of a MEMS pressure sensor, according to the invention.

### DETAILED DESCRIPTION OF THE INVENTION

Before describing the present invention in detail, it is to be understood that this invention is not limited to particularly exemplified apparatus, systems, materials, structures or methods, as such may, of course, vary. Thus, although a number of apparatus, systems, materials, structures and methods similar or equivalent to those described herein can be used in the practice of the present invention, the preferred apparatus, systems, materials, structures and methods are described herein.

It is also to be understood that the invention is not limited to any particular application used herein in connection with a described embodiment of the invention.

Further, the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Where a range of values is provided, it is to be understood that each intervening value, to the tenth of a unit of the lower limit unless the context clearly dictates otherwise, between the upper and lower limit of that range and any other stated or intervening value in that stated range, falls within the scope of the invention. The upper and lower limits of these smaller ranges may independently be included in the smaller ranges and also fall within the scope of the invention, subject to any specifically excluded limit in the stated range. Where the stated range includes one or both of the limits, ranges excluding either or both of those included limits also fall within the scope of the invention.

As used in this specification and the appended claims, the singular forms “a”, “an” and “the” include plural referents unless the content clearly dictates otherwise. As such, this statement is intended to serve as antecedent basis for use of such exclusive terminology as “solely”, “only” and the like in connection with the recitation of claim elements, or use of a “negative” limitation.

Further, all publications, patents and patent applications cited herein, whether supra or infra, are hereby incorporated by reference in their entirety.

The publications, patents and published patent applications discussed herein are provided solely for their disclosure prior to the filing date of the present application. Nothing herein is to be construed as an admission that the present invention is not entitled to antedate such publications, patents and published patent applications by virtue of prior invention.

### DEFINITIONS

The term “a volume”, as used herein, means any space, chamber, cavity, substance, tissue, area or the like.

The term “physiologic”, as used herein, means that in certain embodiments of the invention, the pressure sensors (and associated systems), which are described in detail below, are configured (e.g., shaped, dimensioned, etc.) so that they can be positioned in or on a body of a living organism, e.g., a human.

The terms “substantially fully encapsulating” and “substantially fully covering” are used interchangeably herein and mean a layer or layers or a pouch that covers at least a substantial portion of one or more electrical components and/or a circuit associated therewith.

The term “significantly reduced functionality”, as used in connection with an electronic component or device, means that the functionality or sensitivity of the electronic component or device is not reduced more than approximately 30%.

The term “body”, as used herein, means and includes the body of a human and/or animal.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As summarized above, in certain embodiments, the present invention comprises materials and methods for insulating implantable passive and active electronic components and devices, whereby the electronic components and devices are suitable for prolonged direct body fluid exposure. In certain embodiments, the invention comprises implantable pressure sensors, and associated systems and methods, which provide an accurate and stable sensor output under varying in vivo and ambient conditions, and during prolonged direct body fluid exposure.

It is to be understood that although the materials and methods for insulating implantable passive and active electronic components and devices, in accordance with the invention, are illustrated and described in connection with selective electronic components, and pressure sensor systems and devices, it is emphasized that the materials and methods of the invention are not limited to the illustrated components, systems and devices. Indeed, as will readily be appreciated by one having ordinary skill in the art, the materials and methods of the invention are readily applicable to virtually all electronic components, including passive and active electronic network components, and electronic systems and devices, including medical instruments.

As discussed in detail below, in certain embodiments, the electronic component includes an electrically insulating pouch that substantially fully encapsulates the electronic component. In certain embodiments, the electrically insulating pouch fully encapsulates the electronic component.

In certain embodiments, the insulating pouch comprises a first protective layer and a second electrically insulating layer.

According to the invention, the protective layer can impart physical protection, chemical protection or the like. In certain embodiments, the protective layer also imparts electrical insulation.

In certain embodiments, the first protective layer comprises a compliant (or flexible) polymeric gel, e.g. silicone gel, having appropriate medical grade and purity. According to the invention, the polymeric gel preferably has a low solvent content to provide for thorough curing, whereby optimum dimensional stability is achieved.

According to the invention, the outer surface of the polymeric gel provides a wide surface for the application of an electrically insulating layer thereon. According to the invention, the resultant large diaphragm area can be (an) order(s) of magnitude greater than the component, such as the diaphragm area of a pressure sensor; requiring much less force to displace than would be required for a small diameter coating applied directly to the sensor.

According to the invention, multiple electronic components and/or circuitry associated therewith can be encapsulated with the electrically insulating pouches of the invention.

Referring now to FIG. 1, there is shown an electronic network of a pressure sensor system of the invention. As illustrated in FIG. 1, and discussed in detail below, the sensor network includes a MEMS sensor module 30, having a MEMS pressure sensor 32 associated therewith, an application-specific integrated circuit (ASIC) module 40, and associated circuitry 21, i.e. a pressure sensitive capacitor die wire.

As further illustrated in FIG. 1, in accordance with certain embodiments of the invention, the electronic components, i.e. MEMS pressure sensor module 30 and ACIS module, and at least a portion of the circuitry 21 are fully encapsulated with an electrically insulating pouch 3, whereby the noted electronic components can be exposed to body fluids for prolonged periods without exhibiting significantly reduced functionality.

In certain embodiments, the electronic components, i.e. MEMS pressure sensor module 30 and ACIS module, and at least a portion of the circuitry 21 are substantially fully encapsulated with the electrically insulating pouch 3.

In a preferred embodiment of the invention, the insulating pouch 3 comprises a first protective layer 7 and a second electrically insulating layer 5.

In certain embodiments, the protective layer 7 also comprises an electrically insulating layer.

In certain embodiments, the protective layer 7 comprises a flexible polymeric material.

In certain embodiment, the flexible polymeric material comprises a flexible polymeric gel.

In certain embodiments, the flexible polymeric material comprises a silicone gel.

In certain embodiments, the flexible polymeric material comprises a thermally molded Supersoft® material from Polygel, Inc. (Whippany, N.J.).

In a preferred embodiment of the invention, the protective layer 7 is initially applied to or over the electronic components and preferably cured thoroughly to assure dimensional stability. After the protective layer 7 is adequately cured, the electrically insulating layer 5, i.e. parylene, is applied over the base protective layer 7.

According to the invention, various conventional means can be employed to form or apply the protective layer 7 to the electronic components 30, 40 and circuitry 21. Suitable conventional means include dip coating, molding, laser and etching treatments, spraying, or the like.

Various conventional means can also be employed to apply the electrically insulating layer 5 to or over the protective layer 7. Suitable conventional means include ion-beam deposition, sputtering, dip coating, molding, and laser and etching treatments, and vapor deposition.

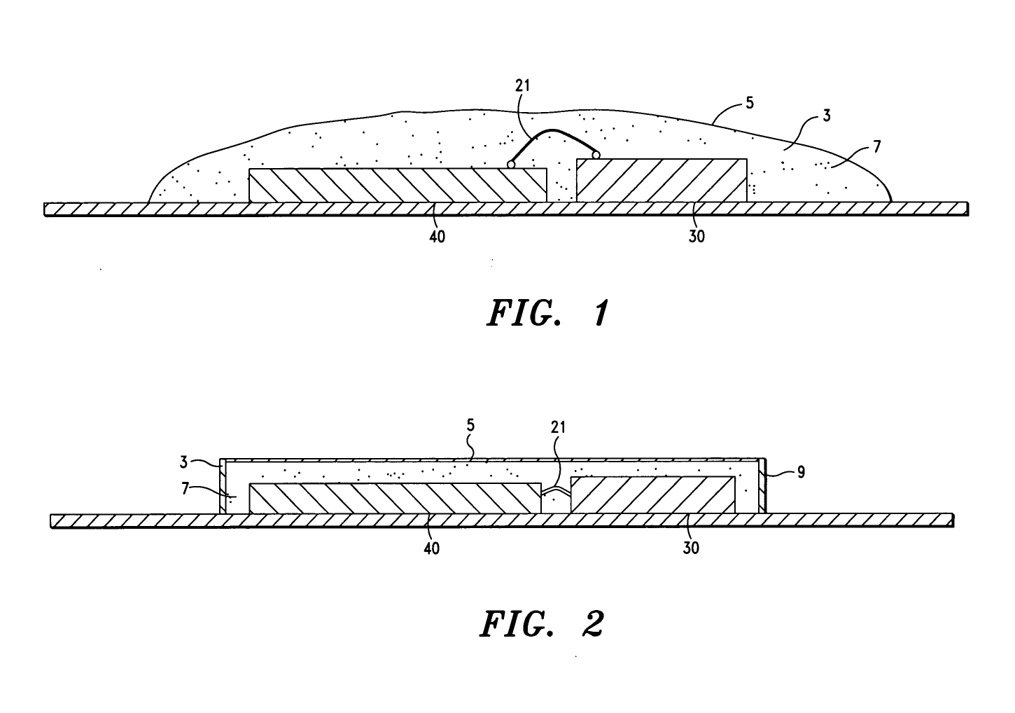

In certain embodiments of the invention, the electrically insulating pouch 3 includes encasement means 9, as illustrated in FIG. 2. According to the invention, the encasement means provides a substantially rigid border for the protective layer 7 and electrically insulating layer 5, whereby the structural integrity of the pouch 3 is enhanced.

According to the invention, the encasement means 9 can comprise various convention materials, including, without limitation, polymeric and ceramic materials. The encasement means can also comprise suitable metals, such as titanium.

In certain embodiments of the invention, the minimum thickness of the protective layer 7 is preferably in the range of approximately 10-500 microns, more preferably, in the range of approximately 50-200 microns.

In certain embodiments of the invention, the maximum thickness of the protective layer 7 is preferably in the range of approximately 50-1000 microns, more preferably, in the range of approximately 100-750 microns.

In certain embodiments of the invention, the thickness of the electrically insulating layer 5 is preferably in the range of approximately 1-10 microns, more preferably, in the range of approximately 2-7 microns.

As discussed in detail below, according to the invention, the electrically insulating pouch 3 can also be applied to or disposed over the entire structure, including either a rigid body or circuit board on which a coil or antenna has been mounted or to a flexible circuit on which the coil or antenna has been mounted. Thus, the entire pressure sensor system, including the pressure module 30, ASIC module 40, and the transmitting/receiving coil can be formed and provided with a moisture proof and protective pouch without significantly diminishing the sensitivity of the pressure sensor.

The improved pressure sensors and associated systems of the invention will now be described in detail. In certain embodiments of the invention, the pressure sensors and associated systems include the systems, components, and functions of the pressure sensors and associated systems disclosed in Co-Pending application Ser. Nos. 12/655,405 and 12/799,752, which, as indicated above, are expressly incorporated by reference herein in their entirety.

Basic pressure sensor operation and telemetry means are also disclosed in U.S. Pat. Nos. 4,186,079, 5,325,865, 6,113,553, 6,285,899, 6,454,720, 6,558,336, 6,731,976 and 6,692,446; each of which is similarly incorporated by reference therein in its entirety.

The pressure sensors of the invention are designed and particularly adapted to accurately measure pressure in a volume over extended periods of time. As indicated above, “a volume”, as used herein, means any space, chamber, cavity, substance, tissue, area or the like. In connection with the pressure sensor embodiments, and associated systems and methods described herein, a volume comprises a chamber of a human body, such as a cranial cavity, but this is only one example of a volume, and the invention is in no way limited to such a chamber. According to the invention, “a volume” can also comprise a space, chamber, cavity or the like that is not in a human body.

The pressure sensors of the invention can also be employed in a wide variety of non-medical contexts. Therefore, although the following discussion generally focuses on measuring pressure in cavities and chambers in a human body, the invention is in no way limited to such application.

As will readily be appreciated by one having ordinary skill in the art, the pressure sensors, and associated systems and methods of invention, provide several significant advantages compared to prior art pressure sensors and methods. A significant feature and, hence, advantage of the pressure sensors of the invention is that they provide very accurate and stable outputs; particularly, when exposed to body fluids. The pressure sensors can thus be positioned in (or on) a body for predetermined periods of time, without significant, if any, functional deterioration.

As set forth in the noted Co-Pending application, base pressure sensors generally include a housing or case, a pressure sensing system, power supply means (or an energy source), and communication means. The pressure sensors also preferably include a temperature and/or drift compensation system.

In certain embodiments of the invention, the pressure sensor systems of the invention additionally include a pressure compensation system.

In certain embodiments of the invention, the pressure sensing systems include at least a first MEMS pressure sensor, which is disposed in the sensor housing. In certain embodiments, the pressure sensing systems include at least two MEMS pressure sensors; at least one MEMS pressure sensor being disposed in the sensor housing and at least one MEMS pressure sensor being disposed at an external position, e.g., at the end of a coupled cable or an external reader.

In certain embodiments of the invention, the sensor housing is adapted to securely position at least one MEMS pressure sensor, and associated components, modules and circuitry, within the sensor housing. In certain embodiments, the housing is further designed and adapted to facilitate placement of the pressure sensor in or on a subject's body.

In certain embodiments of the invention, the communication means includes a communication network or link. In certain embodiments, the communication link comprises a wireless link, i.e. telemetric pressure sensors. In certain embodiments, the communication link comprises conductive wires or similar direct communication means.

In certain embodiments of the invention, the pressure sensors also include at least one additional sensor, preferably, a MEMS sensor. According to the invention, the additional sensor can include, without limitation, a temperature sensor, pO₂ sensor, pCO₂ sensor, and SpO₂ sensor.

In certain embodiments, the pressure sensors of the invention employ selected materials (and associated processing means) and a unique component configuration, which impart a low drift characteristic to the pressure sensor structure. In certain embodiments, the pressure sensors of the invention include a unique digital capacitance system and an ASIC that provides translation from capacitance variation to pressure and individual correction for a calibrated temperature coefficient, which also significantly enhance the accuracy of the sensor output(s) when subjected to varying conditions.

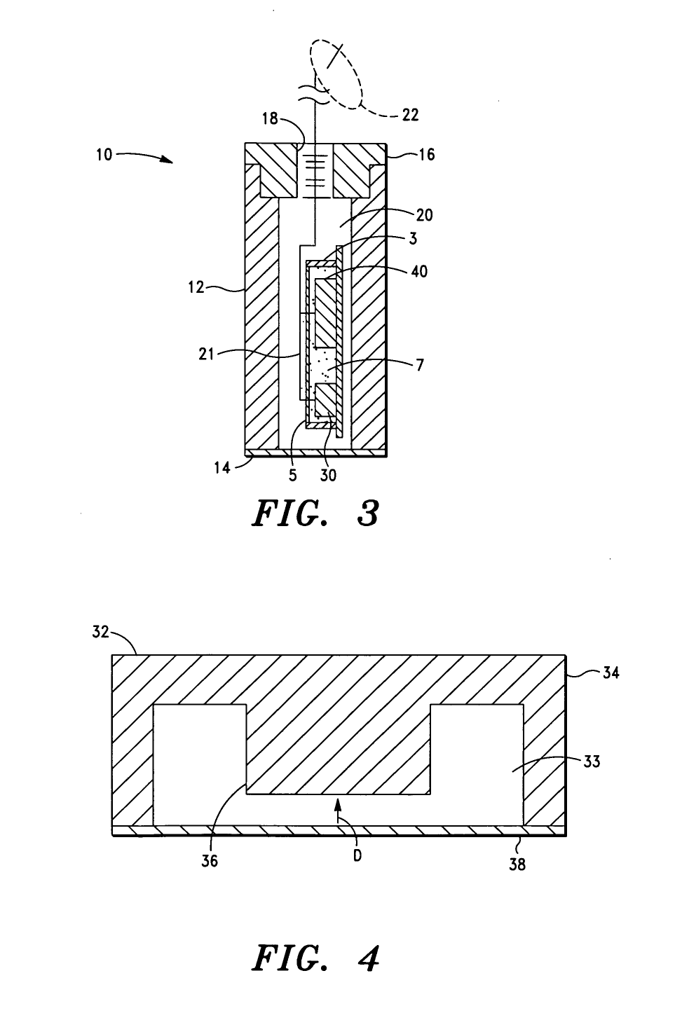

Referring now to FIG. 3, there is shown one embodiment of a pressure sensor 10 of the invention. As illustrated in FIG. 3, the pressure sensor 10 generally includes a housing or case 12, a membrane 14 disposed at a first end, and a cap 16, having a lumen or feed-through 18 therethrough, disposed on a second end.

Disposed within the sensor housing 12 is a sensor module (i.e. pressure sensing system) 30, an ASIC module 40, associated circuitry 21 that facilitates communication by and between the sensor module 30, ASIC module 40 and the communication means, and a pressure transmitting fluid 20, such as silicone oil.

As set forth in Co-Pending application Ser. Nos. 12/655,405 and 12/799,752, in certain embodiments, the ASIC module 40 is designed and adapted to perform at least one of the following functions: (i) compare a variable current to a reference capacitance, (ii) provide signal shaping, (iii) provide pressure sensor signal correction based on calibrated temperature coefficient, (iv) provide power management, (v) provide communications to external circuitry, and (vi) control signal transmissions to/from the pressure sensor 10.

As indicated above, in certain embodiments of the invention, the sensor housing 12 is designed and configured to facilitate placement of the sensor 10 in or on a subject's body. Thus, in certain embodiments, the housing comprises a biocompatible material, such as, without limitation, stainless steel, silicon, titanium and polyetheretherketone (PEEK).

In certain embodiments, the membrane 14 similarly comprises a biocompatible material, such as titanium, stainless steel and silicon. In certain embodiments, the membrane 14 comprises titanium.

As also indicated above, in certain embodiments of the invention, the sensor module 30 includes at least one MEMS pressure sensor. In certain embodiments, the MEMS pressure sensors of the invention comprise absolute pressure sensors that are capacitive and optimized to operate within a range of approximately 700-1300 mbar. The MEMS pressure sensors thus include at least one contact that provides access to a measurement capacitance and, in certain embodiments, a reference capacitance.

Referring now to FIG. 4, there is shown one embodiment of a MEMS pressure sensor 32 of the invention. As illustrated in FIG. 4, the MEMS pressure sensor 32 includes a housing 34, having an internal cavity 33 and a diaphragm (or sensing element) 38. The housing 34 further includes an internal post 36 that is configured and positioned to limit pressure induced deflection of the diaphragm 38 (as shown by arrow “D”).

In certain embodiments, the diaphragm 38 comprises monocrystalline silicon. In certain embodiments, the monocrystalline silicon is metallicized.

To inhibit moisture migration in and to the MEMS pressure sensor 32, the sensor is hermetically sealed. Whereas prior art sensor designs have attempted to incorporate a sensing membrane and moisture resistant seal, the present invention incorporates a totally hermetic seal, e.g. titanium metal and ceramic feed-through, to totally prevent moisture (even in a monomolecular state) from diffusing through the membrane or housing and ultimately degrading the performance of the MEMS pressure sensor.

According to the invention, to further enhance moisture resistance of the MEMS sensor 32, an electrically insulating pouch of the invention can be disposed on the MEMS pressure sensor 32 or entire module 30.

As illustrated in FIG. 3, in a preferred embodiment of the invention, an electrically insulating pouch of the invention is disposed on or over the MEMS pressure module 30, ASIC module 40 and a portion of the circuitry 21, whereby at least the MEMS pressure module 30 and ASIC module 40 are fully encapsulated by the electrically insulating pouch 3. In the illustrated embodiment, the electrically insulating pouch 3 comprises a first protective layer 7 and a second electrically insulating layer 5, as described above (see also FIG. 1).

As stated above, the outer surface of the protective layer 7 preferably provides a wide surface for the application of the electrically insulating layer, i.e. parylene layer 5. According to the invention, the resultant large diaphragm area can be (an) order(s) of magnitude greater than the component, such as the diaphragm 38 of the pressure sensor 32, whereby much less force would be required to displace than would be required for a small diameter coating applied directly to the sensor.

According to the invention, an electrically insulating pouch 3 of the invention can also be applied to the entire structure, including a rigid body or circuit board on which a coil or antenna has been mounted or to a flexible circuit on which the coil or antenna has been mounted. Thus, the entire pressure sensor system, including the pressure module 30, ASIC module 40, and the transmitting/receiving coil can be formed and provided with a moisture proof (and protective) pouch without significantly diminishing the sensitivity of the pressure sensor.

In further envisioned embodiments, an electrically insulating pouch 3 of the invention can also be applied to or over the entire sensor, e.g., sensor 10.

As will readily be appreciated by one having ordinary skill in the art, the pressure sensors, and associated systems and methods of invention provide several significant advantages compared to prior art pressure sensors and methods. Among the advantages are the following:
The provision of implantable electronic components and devices that are suitable for prolonged exposure to body fluids.
The provision of implantable medical systems and devices that are suitable for prolonged exposure to body fluids.
The provision of implantable pressure sensors, and associated systems and methods, which provide accurate and stable sensor output under varying in vivo and ambient conditions, and during prolonged exposure to body fluids.

Without departing from the spirit and scope of this invention, one of ordinary skill can make various changes and modifications to the invention to adapt it to various usages and conditions. As such, these changes and modifications are properly, equitably, and intended to be, within the full range of equivalence of the invention.

What is claimed is:

1. A method for insulating an electronic component, the method comprising:
encapsulating the electronic component with an electrically insulating pouch having a first protective layer and a second electrically insulating layer, said first protective layer comprising a flexible polymeric material, said second electrically insulating layer comprising the parylene layer.

2. The method of claim 1, wherein said electrically insulating pouch fully encapsulates said electronic component.

3. The method of claim 1, wherein said electrically insulating pouch includes encasement means for positioning and enclosing said insulating pouch.

4. The method of claim 3, wherein said encasement means includes a rigid border.

5. The method of claim 1, wherein said flexible polymeric material also comprises an electrically insulating layer.

6. The method of claim 1, wherein said flexible polymeric material comprises a flexible polymeric gel.

7. The method of claim 6, wherein said flexible polymeric gel comprises silicone.

8. The method of claim 1, wherein said flexible polymeric material has a minimum thickness in the range of approximately 10-500 microns.

9. The method of claim 1, wherein said electrically insulating layer has a thickness in the range of approximately 1-10 microns.

10. An implantable electronic component that is suitable for prolonged direct body fluid contact, the electronic component comprising:
an electrically insulating pouch, said insulating pouch encapsulating said electronic component, said insulating pouch comprising a first protective layer and a second electrically insulating layer, said first protective layer comprising the flexible polymeric material, said second electrically insulating layer comprising a parylene layer.

11. The electronic component of claim 1, wherein said electrically insulating pouch fully encapsulates said electronic component.

12. The electronic component of claim 1, wherein said electronic component comprises a passive electronic network component.

13. The electronic component of claim 1, wherein said electronic component comprises an active electronic network component.

14. The electronic component of claim 1, wherein said electrically insulating pouch includes encasement means for positioning and enclosing said insulating pouch.

15. The electronic component of claim 1, wherein said flexible polymeric material also comprises an electrically insulating layer.

16. The electronic component of claim 15, wherein said flexible polymeric material comprises a flexible polymeric gel.

17. A pressure sensor system, comprising:
a sensor assembly having at least one MEMS pressure sensor having a pressure sensing element that is adapted to generate a capacitance variation signal in response to exposed pressure, an application-specific integrated circuit (ASIC) that is adapted to generate a pressure signal with the capacitance variation signal, said pressure signal being representative of the exposed pressure, and network circuitry in communication with said MEMS sensor and ASIC,
said MEMS sensor, said ASIC and at least a portion of said circuitry being fully encapsulated with an electrically insulating pouch, said insulating pouch comprising a first protective layer and a second electrically insulating layer, said first protective layer comprising a flexible polymeric material, said second electrically insulating layer comprising a parylene layer.

18. The system of claim 17, wherein said sensor assembly includes a temperature compensation system that is adapted to correct for temperature induced variations in said pressure sensor signal, a drift compensation system that is adapted to correct for pressure and temperature induced drift of said pressure sensor signal, and a pressure compensation system that is adapted to correct for variations in measured pressures of said MEMS pressure sensor and atmospheric pressure.

19. The system of claim 17, wherein said electrically insulating pouch includes encasement means for positioning and enclosing said insulating pouch.

20. The system of claim 17, wherein said flexible polymeric material also comprises an electrically insulating layer.

21. The system of claim 20, wherein said flexible polymeric material comprises a flexible polymeric gel.
